(19) United States
(12) Patent Application Publication
(10) Pub. No.: US 2019/0046563 A1
(43) Pub. Date: Feb. 14, 2019

(54) NANOBUBBLE-CONTAINING COMPOSITION AND USE THEREOF

(71) Applicant: Aqua Zest Corporation, Kawasaki-shi (JP)

(72) Inventor: Ichiro KAMEI, Kawasaki-shi (JP)

(21) Appl. No.: 16/161,192

(22) Filed: Oct. 16, 2018

Related U.S. Application Data
(63) Continuation of application No. 15/193,176, filed on Jun. 27, 2016, now abandoned.
Application No. 15/193,176 is a continuation of application No. PCT/JP2015/050073, filed on Jan. 5, 2015.

(30) Foreign Application Priority Data
Dec. 27, 2013 (JP) 2013-273610
Oct. 17, 2014 (JP) 2014-212676

(51) Int. Cl. A61K 33/00; A23P 30/40; A23L 2/54; A01N 1/02; A61K 9/00; A61K 9/50; A01G 31/00; C12N 5/00; A23L 33/00; A23L 33/10

(52) U.S. Cl. CPC A61K 33/00; A23P 30/40; A23L 2/54; A01N 1/0221; A61K 9/0095; A61K 9/0019; A61K 2300/00; A61K 9/5015; A01G 31/00; C12N 5/0018; A23L 33/00; A23L 33/10

(57) ABSTRACT

A composition contains nanobubbles containing hydrogen, oxygen, and nitrogen, and the use thereof. A composition containing, as an active ingredient, nanobubbles having a diameter of 30 micrometer or less and containing 0.45-0.55 ppm of hydrogen, 10-12.5 ppm of oxygen, and 7-8 ppm of nitrogen. A mitochondria-activating composition, cell growth promoting agent, cell preservation liquid or cryopreservation liquid containing this composition, and a method for using this composition, agent, or liquid. A food or beverage, or a food or beverage raw material, produced using this composition.

### TECHNICAL FIELD

The present invention relates to composition containing nanobubbles and use thereof. More specifically, it relates to composition activating mitochondria and use thereof.

### BACKGROUND ART

Method such as the patent literature 1 is disclosed for example, as a method to dissolve a gas into a liquid using microbubble, nanobubble technology and to promote proliferation of cultured cells. However, Patent Literature 1 neither specifically nor precisely discloses, for example, how large microbubbles or nanobubbles are and what are their composition; and when these microbubbles or nanobubbles are used, what kinds of cells proliferate and how much their proliferation is promoted. Hence, a more practical composition, method, and/or device should be developed. In addition, more efficient proliferation promoting method is also desired.

Hydrogen water, hydrogen-oxygen water are known as products mixing gases using microbubbles and nanobubbles into drink water (for example, patent literature 2). However, these drinking water is not so popular because a limited number of people feel sufficiently satisfactory effect. Therefore, drinking water containing microbubbles and/or nanobubbles which realize effect sufficiently is expected. In addition, drinking water containing microbubbles and/or nanobubbles with new feature has been desired.

On the other hand, mitochondria is an organelle to generate energy and it can be thought that by increasing mitochondria activity, energy production increases to make more active body. However, drinking water which activates mitochondria is not still developed and development of drinking water activating mitochondria is desired.

In addition, there are problems that viability rate decreases at cryopreservation of cells, that contents leaks by cell disruption to worsen texture or flavor. Therefore, development of cell preservation liquid to raise viability and to make a flavor better is desired.

### PRIOR ART DOCUMENTS

### Patent Literatures

Patent literature 1: Japanese patent application No. 2009-234683 bulletin

Patent literature 2: Japanese patent application No. 2008-56741 bulletin

### SUMMARY OF INVENTION

### Problems to be Solved by the Invention

The present invention provides composition containing nanobubbles containing hydrogen, oxygen and nitrogen and use thereof.

### Means to Solve Problem

The specification provides the following inventions.

(1) Composition containing microbubbles and/or nanobubbles with 30 micrometer or less in diameter containing 0.45-0.55 ppm of hydrogen, 10-12.5 ppm of oxygen and 7-8 ppm of nitrogen. Here, concentration of each gas means concentration combining both dissolved gas and nanobubbles.
(2) Composition for activating mitochondria containing the composition of (1) as an active ingredient. Here, composition for activating mitochondria means the same meaning as activating agent of mitochondria.
(3) An agent for promoting cell proliferation containing composition according to (1) or (2) as an active ingredient. In this case, activating agent for cell proliferation may contain both composition according to (1) and (2).
(4) Cell preservation liquid containing any one of compositions or agents according to any one of (1) to (3) as an active ingredient. Here, agent means the activating agent for cell proliferation according to (3). However, agent for activating mitochondria will be included if the composition for activating mitochondria according to (2) will be amended to agent for activating mitochondria. In addition, “any one of” does not mean “only any one of” but means “at least one of”. Therefore, more than 2 or 3 of compositions or agents according to any one of (1) to (3) are included in the technical scope. Situation is the same as followings and “any one of” means “(at least) any one of”.
(5) Cryopreservation liquid containing any one of composition or agent according to any one of (1) to (4) as an active ingredient. Here, agent is the same meaning of (4) and liquid means cryopreservation liquid according to (4).
(6) Food and drink containing any one of composition, agent or liquid according to any one of (1) to (5). Here, the liquid includes cryopreservation liquid according to (5) in addition to cell preservation liquid according to (4).
(7) Food and drink manufactured using materials treated with any one of composition, agent or liquid according to any one of (1) to (5).
(8) A method for activating mitochondria using any one of composition, agent or liquid according to any one of (1) to (5).
(9) A method for promoting cell proliferation using any one of composition, agent or liquid according to any one of (1) to (5).
(10) A method for preserving a cell using any one of composition, agent or liquid according to any one of (1) to (5).
(11) A method for cryopreservation of a cell using any one of composition, agent or liquid according to any one of (1) to (5).
(12) A manufacturing method of food and drink comprising a step for treating food and drink or raw materials thereof using any one of composition, agent or liquid according to any one of (1) to (5).
(13) A medium for cell culture prepared using a composition according to (1).
(14) Solution for plant hydroponics prepared using a composition according to (1).
(15) A method for growing an animal cell using a medium according to (13).
(16) A method for proliferating an animal cell according to (15), wherein the animal cell is an immune system cell.
(17) An agent for prevention or treatment of arteriosclerosis comprising the composition according to (1) or (2) or the agent for promoting cell proliferation according to (3) as an active ingredient.
(18) An agent for prevention or treatment of diabetes comprising the composition according to (1) or (2) or the agent for promoting cell proliferation according to (3) as an active ingredient.
(19) An injection solution comprising the composition according to (1) or (2).

### Effect of the Invention

According to the composition of the present invention, an effect for activation of mitochondria, promotion of cell proliferation, cell preservation, and decreasing damage to a cell at the time of cryopreservation.

### BRIEF DESCRIPTION OF THE DRAWINGS

FIG. 1 is a figure showing promotion effect of proliferation of cell culture by the composition of the present invention.

FIG. 2 is a figure showing antioxidative action of the composition of the present invention.

FIG. 3 is a figure showing influence of various drugs on ATP production.

FIG. 4 is a figure showing change of GPT before and after taking the composition of the present invention.

FIG. 5 is a figure showing production rate of HEL before and after taking the composition of the present invention.

FIG. 6 is a figure showing change of STAS before and after taking the composition of the present invention.

FIG. 7 is a figure showing proliferation promoting effect of cell culture of ES cells by the composition of the present invention.

FIG. 8 is a figure showing distribution rate of various cells in lymphocyte by FACS analysis.

FIG. 9 is a figure showing proliferation promoting effect of T cells, B cells and NK cells at the time of adding MCP of the present invention by ratio assuming 1 as control plot.

FIG. 10 is a figure showing proliferation promoting effect of the composition of the present invention on immunocytes.

FIG. 11 is a figure showing proliferation promoting effect of the composition of the present invention on immunocytes.

FIG. 12 is a figure showing proliferation promoting effect of the composition of the present invention on immunocytes.

FIG. 13 is a figure showing proliferation promoting effect of the composition of the present invention on immunocytes.

FIG. 14 is a figure showing effect of the composition of the present invention on arteriosclerosis.

FIG. 15 is a figure showing effect of the composition of the present invention on arteriosclerosis.

FIG. 16 is a figure showing the effect of the composition of the present invention on type II diabetes.

FIG. 17 is a figure showing the result of blood test after taking MCP.

FIG. 18 is a figure showing proliferation promoting effect of the composition of the present invention on fibroblast.

FIG. 19 is a figure showing inflammatory curing effect of the composition of the present invention.

### DETAILED DESCRIPTION OF PREFERRED EMBODIMENTS

The present invention provide composition containing as an active ingredient, microbubbles and/or nanobubbles having a diameter of 30 micrometer or less and containing 0.45-0.55 ppm of hydrogen, 10-12.5 ppm of oxygen and 7-8 ppm of nitrogen. The composition can be utilized for activation of mitochondria, promotion of cell proliferation, preservation of a cell, cryopreservation of a cell, food and drink, and preparation of injection solution etc.

Preferable concentration of gases contained in the composition of the present invention is hydrogen 0.1 to 3.0 ppm, oxygen 5 to 20 ppm, and nitrogen 3 to 20 ppm, more preferable, hydrogen 0.3 to 1.0 ppm, oxygen 7 to 15 ppm and nitrogen 4 to 15 ppm, more preferable, hydrogen 0.4 to 0.6 ppm, oxygen 9 to 13 ppm and nitrogen 5 to 10 ppm, more preferable, hydrogen 0.45 to 0.55 ppm, oxygen 10 to 12.5 ppm, and nitrogen 7 to 8 ppm. As well, as each concentration has an effect depending on it at each concentration, within these concentrations, effect of the present invention is provided even if it is divided at any concentration. As well, ppm means part per million and represents concentration of a soluble gas.

About gas contained in the composition of the present invention, it is preferable larger than 0 and lower than 30 micrometer in particle diameter, more preferable, lower than 20 micrometer, more preferable, lower than 10 micrometer, more preferable, lower than 1 micrometer, more preferable, lower than 100 micrometer, more preferable lower than 30 nanometer.

In plant hydroponics etc., proliferation efficiency is promoted by using microbubble gas. In that case, as size of microbubbles

1 to 100 micrometer is preferable, more preferable, 5 to 80 micrometer, more preferable, 10 to 70 micrometer, more preferable, 15 to 60 micrometer, most preferable, 20 to 50 micrometer. These do not mean all microbubbles or nanobubbles are included within these sizes but more than 60% of all microbubbles or nanobubbules should be within these sizes.

The composition containing nanobubbles of the present invention is produced by mixing hydrogen gas, oxygen gas and nitrogen gas which are made nanobubbles into water (for example, ultrapure water). Purity of gases is not specifically limited to but it is desirable to use high purity gases for use of drinking water etc.

Nanobubbles can be generated by shearing and destroying gas incorporated into liquid to become minute etc. for example and various nanobubble generation machines are developed. In the present invention, as nanobubble generator, general nanobubble generator can be used with no special limitation but nanobubble generator Buvitas (trademark, Ligaric co. Ltd.) or nanobubble generator Nanoaqua (trademark, Tech corporation co. Ltd.) etc. can be preferably used.

Form of the composition containing nanobubble of the present invention is not specifically limited to, but typically water, drinking water, water for preparation of medium, water for a fish corf and sprinkling water for plant etc.

Structure of apparatus to produce functional water (nanobubble water) contains followings. A hydrogen gas cylinder, an oxygen gas cylinder, a nitrogen gas cylinder or a nitrogen gas generator, a digital control type regulator, an ozone generator, nanobubble generator, water supply tank, and various filter groups.

At first, using about 1 ppm ozone water, a nanobubble generator, a water supply tank and pipes are washed and sterilized. When fresh water is used as raw water of functional water, structure of filter to be passed is an array of a hollow fiber membrane filter, an activated carbon filter, and a hollow fiber membrane filter in this order sequentially which is used as one unit. If tap water is raw water, usually two units are used.

Number of units can be increased or decreased appropriately according to degree of impurities of raw water. The filtrated water which passed the filter unit is passed through 0.25 to 0.45 micrometer membrane filter (a reverse osmosis membrane filter according to object) and is further filtrated to pour into water supply tank.

Gas with adjusted concentration by a digital control type regulator is send from each tank etc. to a nanobubble generator and dissolved until each gas reaches to the numerical value of desired dissolved amount with circulation of each gas and nanobubble to filtrated water in a water supply tank between a nanobubble generator and a water supply tank. Materials in state that gas in water reaches to desired dissolved amount by various inspection apparatus is used by filtration through 0.25 to 0.45 micrometer membrane filter from a water supply tank.

The present invention provides an activating agent which activates mitochondria. Activation of mitochondria is measured by ATP production as increase of ATP production.

In this specification, “activation of mitochondria” means that ATP production in mitochondria becomes higher than that of control plot. Control plot means typically ordinary water (ultrapure water), but not limited to these and means experimental plot using the composition of the same composition other than not containing nanobubbles of the present invention. Using the composition of the present invention has an effect that ATP production amount becomes higher than control plot at the time of loading oxidative stress.

According to the composition of the present invention, high promotion effect of cell proliferation is obtained. For example, when medium for cell culture using the composition (solution) of the present invention is prepared to culture cells, higher promotion effect of growth than a medium using ordinary water (ultrapure water).

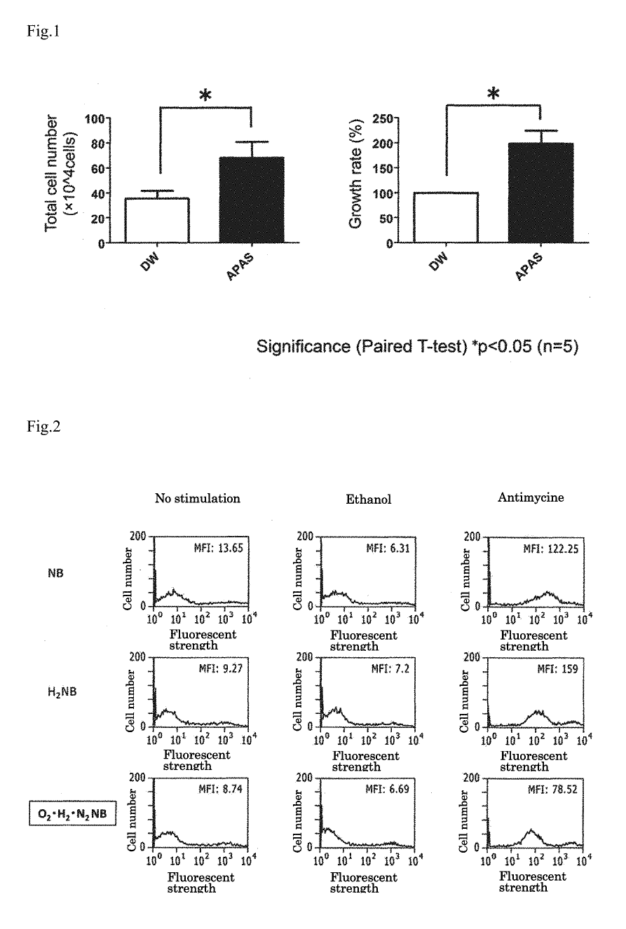

For example, in culture of iPS cells (induced pluripotent stem cells), double promotion effect of growth was obtained at 72 hr culture compared with culture in a medium using ordinary ultrapure water (FIG. 1). The promotion agent for cell growth can be used for other cultured cells, for example, primary culture cells, undifferentiated cells, a hematopoietic cell, a fibroblast, an immortalized cell, a stem cell for the cell which can proliferate. Here, it is not limited in particular, but the cultured cell means the cells which can be divided such as a primary somatic cell, an established cell, an immortalized cell, a stem cell etc. A stem cell includes an embryonic stem cell and a somatic stem cell and means a neural stem cell, a liver stem cell, an epidermal stem cell, a germ cell and an iPS cell etc. but not limited to these. A cell which can proliferate means a cell which has ability to proliferate and includes a cultured cell of a primary somatic cell, an undifferentiated cell, and a progenitor cell etc.

Any cells other than germ cells of mammalian origin (e.g. mice or humans etc.) can be used as starting material for the production of cultured cells. For example, keratinizing epithelial cells (e.g., keratinized epidermal cells), mucosal epithelial cells (e.g., epithelial cells of the superficial layer of tongue), exocrine gland epithelial cells (e.g., mammary gland cells), hormone-secreting cells (e.g., adrenomedullary cells), cells for metabolism or storage (e.g., liver cells), intimal epithelial cells constituting interfaces (e.g., type I alveolar cells), intimal epithelial cells of the obturator canal (e.g., vascular endothelial cells), cells having cilia with transporting capability (e.g., airway epithelial cells), cells for extracellular matrix secretion (e.g., fibroblasts), constrictive cells (e.g., smooth muscle cells), cells of the blood and the immune system (e.g., T lymphocytes), sense-related cells (e.g., bacillary cells), autonomic nervous system neurons (e.g., cholinergic neurons), sustentacular cells of sensory organs and peripheral neurons (e.g., satellite cells), nerve cells and glia cells of the central nervous system (e.g., astroglia cells), pigment cells (e.g., retinal pigment epithelial cells), progenitor cells (tissue progenitor cells) thereof and the like are listed. There is no limitation on the degree of cell differentiation, even is undifferentiated progenitor cells (including somatic stem cells) and finally differentiated mature cells can be used alike as sources of somatic cells in the present invention. Examples of undifferentiated progenitor cells include tissue stem cells (somatic stem cells) such as neural stem cells, hematopoietic stem cells, mesenchymal stem cells, and dental pulp stem cells.

In addition, by using the composition of the present invention, promotion effect of plant cell proliferation is obtained. Promotion effect of proliferation is obtained by preparing a medium or solution for hydroponic culture and by using for plant. Especially, in hydroponic culture, by adding air microbubbles (size 10 nm to 30 nm) in addition to the composition of the present invention, dry weight of part above the ground, fresh weight of part above the ground, dry weight of underground part, and fresh weight of underground part increased by 10 to 20% (refer to Example 14, table 7).

According to the composition of the present invention cells can be preserved with state of keeping high cell activity. For example, when fishes are grown in water containing the composition of the present invention and preserved, activity of mitochondria can be kept high, and food with good flavor can be provided. This is thought to be caused by increase of ATP production with activation of mitochondria to increase taste component such as inosine acid (IMP) which is degradation product of ATP etc.

The composition of the present invention may be added to food and drink, used for raw material of food and drink, and also used for the processing of food and drink or food and drink raw materials. Food and drink is not limited to but means food and drink which human takes. For example, a healthy supplement, a low calorie food, diet food, bread, dairy products (e.g., no fat milk, yogurt, a pudding, soybean milk), noodles (e.g., udon, Chinese noodles, a won ton, a coating dough for jiaozi, buckwheat noodle), a cake (e.g., a cookie, a Japanese sweet, steamed bread, potato chip, chocolate, custard cream), drink (e.g., soup, vegetables drink, a diet drink, tea, coffee), dessert (e.g., a cake, a mousse), a frozen confectionery (e.g., jelly, ice cream), a side dish (e.g., minced fish, a rolled omelet, fried chicken), a cereal, filling, dressing, fried food, processed meat (e.g., sausage) are listed.

The composition of the present invention can be used to add feedstuff. A feedstuff is not limited to but means animals excluding human, preferably, food and drink for fishes, livestock and pet etc. In addition, feed for fish is included. More preferably, water of fish corf, water tank and pond etc. and food and drink for cattle, pigs, sheep, chickens, horses, dogs, and cats etc.

In addition, the composition of the present invention can be used for preparation for injection solution (including intravenous drip). For example, it can be used as proliferation promotion effect of immunocytes and preventive and/or therapeutic drugs by injecting the composition of the present invention to pet, livestock, experimental animal and/or human etc. In addition, the composition of the present invention can be used to prepare injection.

In addition, the composition of the present invention provides promotion effect of immune cell proliferation, and preventive and therapeutic effect for diabetes, arteriosclerosis, and inflammation etc.

The present inventions are explained as follows using examples but the scope of the present invention is not limited to by the examples.

### EXAMPLES

### Example 1

Medium for iPS cells is prepared as follows. 12.5 g of Glasgow minimum essential medium (Sigma; G6148) and 27.5 g of Sodium bicarbonate (Sigma; S5761) are added to 1 L of distilled water (DW) or to the composition of the present invention and stirred. To the solution, each reagent is added to the following final concentrations: 0.1 mM MEM Non-Essential Amino Acids Solution (Gibco; 11140), 1 mM Sodium pyruvate solution (Sigma; S8636), 1% Fetal bovine serum (CCB; 171012), 1% PenicillinStreptomycin (Gibco; 15140), 5.4% Knockout Serum Replacement (Gibco; 10828), 0.1 mM 2-Mercaptoethanol (Wako; 137-06862), and 2000 U/ml Leukemia inhibitor factor (Millipore; ESG1107). Cell maintaining medium is prepared by stirring to homogeneous, dissolved, and filter-sterilized.

The result of culture is shown in FIG. 1. As a result, between the case that medium was prepared using the composition of the present invention and the case that medium was prepared using ordinary ultrapure water, 2 fold growth yield is obtained in the medium prepared using the composition of the present invention compared to the control ultrapure water medium.

### Example 2 Study of Anti-Oxidant Action Using Mouse Vascular Endothelial Cells (MAEC)

Culture medium is prepared by making M199 medium (Sigma) using water containing the composition of the present invention, nanobubble water (nanobubble water containing air) or hydrogen nanobubble water (hydrogen as dissolved gas, nanobubble water containing more than 70% of sum of microbubbles and nanobubbles) respectively and by adding 10% fetal bovine serum, 100 IU/ml penicillin and 100 micrometer of streptomycin. After MAEC was cultured for 24 hrs with medium using each water respectively, it was cultured further 2 hrs. After that, 30 microgram/ml antimycin was added to each medium and it was cultured for 30 min. As a control, ethanol which was used as solvent was added at the same concentration, 0.06 v/v %, as a dosage of antimycin solution. Antimycin is known to have an effect producing ROS (Reactive Oxygen Species) to cells by suppressing electron transport system of mitochondria. Ethanol is generally used as a solvent of antimycin and to know the effect of the solvent it was tested about the case that only ethanol was added without antimycin. To know produced ROS quantity, number of cells having the fluorescent strength was counted using fluorescence intensity as an index. Specifically, CM-H2DCFDA was used as a probe to detect ROS and fluorescence intensity from each cell is detected by flow cytometer (Vantage SE, Becton Dickinson).

Results of ROS detection is shown in FIG. 2. Each graph shows cell number showing some fluorescence intensity and the value of MFI shows average fluorescence intensity. When MAEC cells were cultured in the medium using nanobubble water (NB; upper column), almost no change was observed about average fluorescence intensity at the time of no stimulation (left column) and at the time that ethanol was added as solvent control (middle column). However, average fluorescence intensity was significantly increased at the time of adding antimycin having ROS producing action.

In contrast, when cultured in the water containing the composition of the present invention (O2.H2.N2NB; lower part), lower average fluorescence intensity was obtained in either case of no stimulation and adding antimycin than cultured in a medium using nanobubble water. In addition, when compared among the case being cultured in the medium using hydrogen nanobubble water (H2NB; middle column) and the case being cultured in the medium using the water containing the composition of the present invention, and the case being cultured in the medium using the water containing the composition of the present invention showed lower average fluorescence intensity in all cases of no stimulation, adding only ethanol, and adding antimycin. From these results, strong anti-oxidative effect of the water containing the composition of the present invention was observed.

### Example 3 Investigation of ATP Production in Mouse Vascular Endothelial Cell Line (MAEC) at the Time of Loading Oxidative Stress

Next, intracellular ATP production was investigated to confirm action of ATP producing active water to mitochondria function which decreases at the time of loading oxidative stress in MAEC. MAEC cells were cultured in the culture medium prepared using water containing the composition of the present invention, nanobubble water and hydrogen nanobubble water as above experiment. After MAEC was cultured in medium using respective water, medium was changed with fresh medium using respective water and it was cultured for 2 hrs. After that, 50 microgram/ml antimycin, 0.06 v/v % ethanol and 500 micromol/L hydrogen peroxide were added to respective medium. In addition, as a control of anti-oxidative action, anti-oxidative agent, N-acetyl cysteine (NAC) was used which acts on a wide range of reactive oxygen species.

A medium for control of anti-oxidative action was prepared using ordinary water.

In this medium, MAEC cells were cultured for 24 hrs, medium was changed to fresh medium using ordinary water and cultured for further 2 hrs. After that, NAC was added to final concentration of 5 mmol/L 30 minutes before treatment of antimycin or hydroxyl peroxide. Then, change of ATP quantity in cells was measured by luciferase activity as index (ATPlite, PerkinElmer).

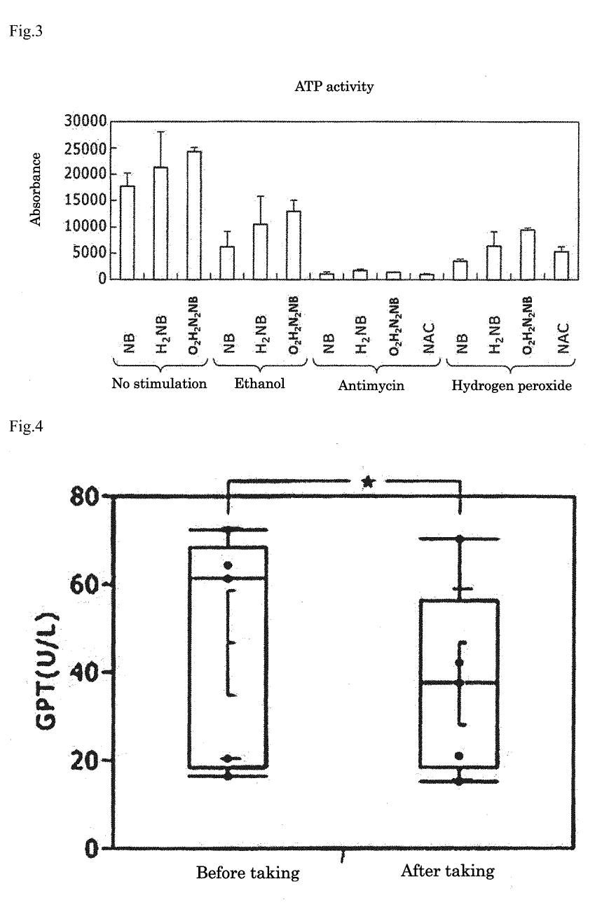

Results of ATP quantity measurement in cells is shown in FIG. 3. In the cells cultured in medium using nanobubble water (NB), ATP quantity decreased in all the treatment of ethanol, antimycin and hydroxyl peroxide compared to no stimulation.

From these results, ethanol, antimycin and hydroxyl peroxide have suppression effect of ATP producing activity.

Stronger decrease of ATP producing activity was observed in antimycin treatment than in treatment of hydroxyl peroxide. Cells cultured in medium using hydrogen nanobubble water (H2NB) and water containing the composition of the present invention (O2.H2.N2NB respectively, showed increase of ATP producing activity in either case. That is, water containing hydrogen nanobubbles and the water containing the composition of the present invention decreased the suppression activity of ATP producing activity by antimycin etc. equal or more extent as or than anti-oxidative agent, NAC. Furthermore, the water containing the composition of the present invention significantly reduced the suppression effect of ATP producing activity in case of treatment of ethanol or hydroxyl peroxide than hydrogen nanobubble water.

### Example 3 Investigation of Drinking Effect on Human

Drinking experiment on human was carried out to elucidate the effect on living body when human drinks the water containing the composition of the present invention. Human joined to the experiment are 4 men and 1 woman of 40 to 69 years old (average age 51.8). Age and sex of each participant are shown in Table 1. All participants are in healthy state.

TABLE 1

| | | Age |
|---|---|---|
| | Sex | (year) |
| 1 | male | 40 |
| 2 | male | 69 |
| 3 | male | 50 |
| 4 | femal | 47 |
| 5 | male | 53 |

Each participant drink 1 L/day of the water containing the composition of the present invention for 4 weeks and is about to live usual life. Blood of participants was drawn before drinking start and blood was drawn again after 4 weeks after drinking start. They don't drink the water containing the composition of the present invention on the day of blood sampling. Test was carried out about GPT (glutamic pyruvic transaminase) which is indicator of liver disorder, HEL (hexanoyl-lysine) which is marker of lipid peroxide and STAS (serum total antioxidation status) which is known as soluble total antioxidative substance.

(1) GPT

GPT is an enzyme which exists mainly in liver. As it leaks to blood specifically when liver cells were destroyed, it is used as index of liver disorder by hepatitis virus or drugs etc. In addition to this, fatty liver has a little disorder about GOT and GPT value in many cases, fatty liver due to obesity is known to have higher GPT than GOT. About GPT, concentration in serum was measured sing silica liquid ALT reagent (Kanto chemical co., Ltd), BioMajesesty (JCA-BM8060) (JEOL Ltd.) and Olympus AU5431 type biochemical autoanalyzer (Olympus co., Ltd.). Serum was separated and recovered by coagulation of collected blood and by centrifugation. Results of measurement are shown in FIG. 4 by box plot. Referring to FIG. 4, GPT value apparently decreased (P=0.042) after taking the water containing the composition of the present invention compared to before drinking. Therefore, tendency of fatty liver improvement was observed by drinking the water containing the composition of the present invention.

(2) HEL

HEL is a stable early product derived from fatty peroxide (13-hydroperoxy-octadecadienoic acid, 13-HPODE) in lipid peroxidation process by active oxygen species. Different from traditionally used aldehyde based lipid peroxidation marker such as 4-HN of MDA etc., the marker can catch an early stage of lipid peroxidation HEL concentration in serum was measured using ELISA kit for hexanoyl lysine (KHL-700, NIKKEN SELL Co., Ltd.) and using full automatic microplate EIA analyzer AP960 (Kiowa medics co. Ltd.). Results of measurement are shown in FIG. 5 in box plot. Referring to FIG. 5, HEL decreased after drinking ATP production activation water (P=0.028). Therefore, the water containing the composition of the present invention is shown to have suppressing effect of lipid peroxidation in human.

(3) STAS

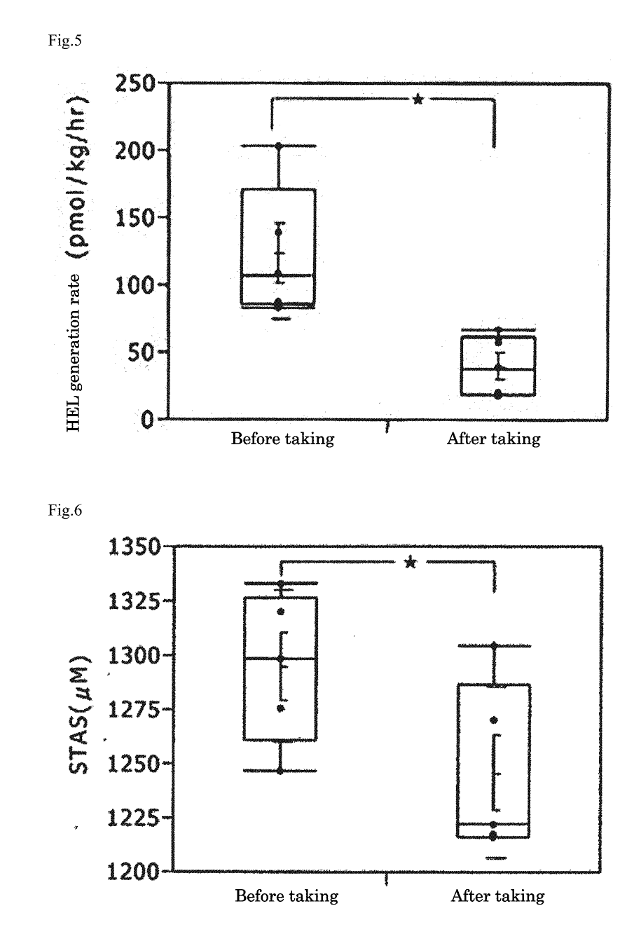

STAS is a soluble antioxidative substance and enables measurement of total antioxidative activity to oxidative stress. STAS was measured about serum using TOTAL ANTIOXIDATION STATUS (NX2332, RANDOX Laboratories Ltd) and using 7020 type Hitachi autoanalyzer (Hitachi High-Technologies Corporation). Results of measurement are shown in FIG. 6 by box plot. Referring to FIG. 6, STAS showed significantly low value after taking (P=0.042). From these results, as STAS estimates soluble antioxidative substances totally, it is speculated that the water containing the composition of the present invention acts on reduction of hydroxyl radicals or active oxygen species other than peroxinitrite which is expected to be scavenged by the water containing the component of the present invention.

### Example 4

2 subjects (O, I) was made to take 500 ml of the water containing the composition of the present invention (APAS, O2.H2.N2 NBwater, also called MCP) a day for 1 month and had a blood test before and after that.

Results

Subject O

Compared with the value before taking the water containing the composition of the present invention, after taking it, ALP (alkaline phosphatase), ALT and gamma-GTP were improved. In addition, values of total cholesterol, LDL cholesterol, and free fatty acid were also improved.

Subject I

Compared with the value before taking the water containing the composition of the present invention, after taking it, values of ALP (alkaline phosphatase), ALT and gamma-GTP were improved. In addition, value of total cholesterol, LDL cholesterol, free fatty acid and neutral fat are also improved.

Judged by the results of the two people comprehensively, by taking the water containing the composition of the present invention, liver function was improved and clinical examination value related to lipid is thought to be improved.

TABLE 2

| | |
|---|---|
| subject O | subject I |

| | before | after | before | after |
|---|---|---|---|---|
| | drinking | drinking | drinking | drinking |
| ALP | 183 | 171 | 265 | 248 |
| ALT | 56 | 54 | 63 | 54 |
| γ-GTP | 182 | 151 | 90 | 80 |
| total cholesterol | 147 | 105 | 117 | 96 |
| LDL cholesterol | 147 | 105 | 117 | 96 |
| free fatty acid | 1.2 | 0.47 | 0.85 | 0.82 |
| neutral fat | 156 | 172 | 192 | 179 |

### Example 5

In Vitro Experiment on Growth Rate of ES Cells

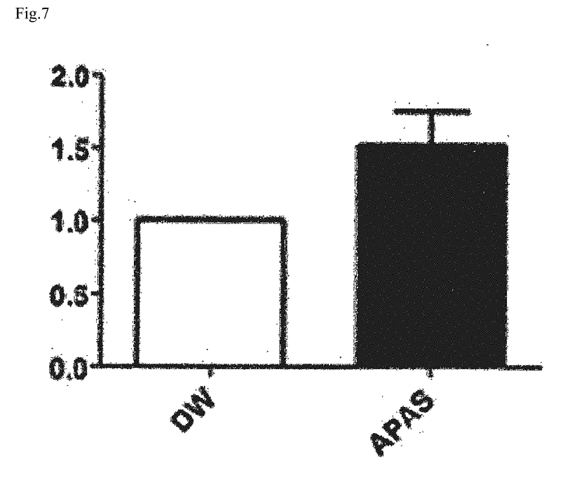

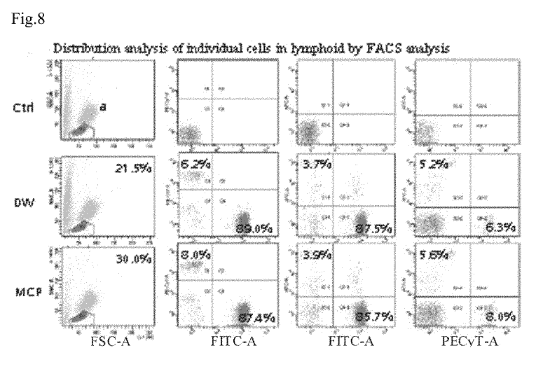

When growth of mouse ES cells in media prepared using DW or APAS (the same composition as iPS cell medium in Example 1) was examined, about 1.5 fold of growth rate was observed when medium was prepared using APAS in case that growth rate of 3 days culture wad assumed 1 (FIG. 7),

### Example 6

### In Vivo Experiment on Proliferation Promotion of Immunocyte (T Cells, B Cells, NK Cells) by MCP

### Type of Water Added to Medium

### Experiment A

(1) raw water (raw water for preparing MCP)
(2) raw water+gases (without filtration)
(3) raw water+gases+0.22 micrometer filter

Method for rearing a rat As an experimental animal, SD rat (12 w) male was used. Method to give water to a rat is carried out by both oral administration and intraperitoneal administration. Intraperitoneal administration was carried out by intraperitoneal injection. MCP filtrated with 0.22 micrometer filter was used for oral administration. On the other hand, for control group, DW fixed is administered. Intraperitoneal administration was done using MCP or DW for culture which is diluted to 1 fold to 10 fold PBS (1 ml×twice a day). Water change and injection was carried out twice, morning and night.

Experiment period On 3^{rd} day and 7^{th} day after start of the experiment estimation was carried out. Control group (n=2) and test group (n=2) were used for 1 assessment.

Experimental Method

sampling from a rat heart (4 ml) was carried out and blood was put into 15 ml tube. To this tube, ⅕ volume of HetaSep (800 microliter) was added and mixed well and incubated (37 C, over 2 hrs). After that, confirmation of separation of a red blood cell layer was done. After 2 hrs, lymphocyte ingredient coagulated around the tube and red blood cell ingredient at the center of tube and about 2 ml of lymphocyte ingredient was separated by a pipette at this time. After that, lymphocyte ingredient was recovered to 15 ml tube by 2 ml/sample and centrifuged (3000 rpm, 8 min, RT). It was adjusted to 30 microliter/sample with PBS (RT) and dispensed 30 microlitter/tube in eppentube and rest was used as control. IOTest (30 microlitter) was added to each tube and pipetting was done (SampleIOTest=1:1). After that, antibody reaction is carried out 20 min/RT, washed with FACS buffer (1×), and centrifuged (3,000 rpm, 5 min., RT). Sup was removed and VersaLyse (500 microlitter/tube) was added under shielding light and stand for 10 min. at RT. After that, counting of total cell number and measurement of immunocyte ratio by FACS were carried out.

Results

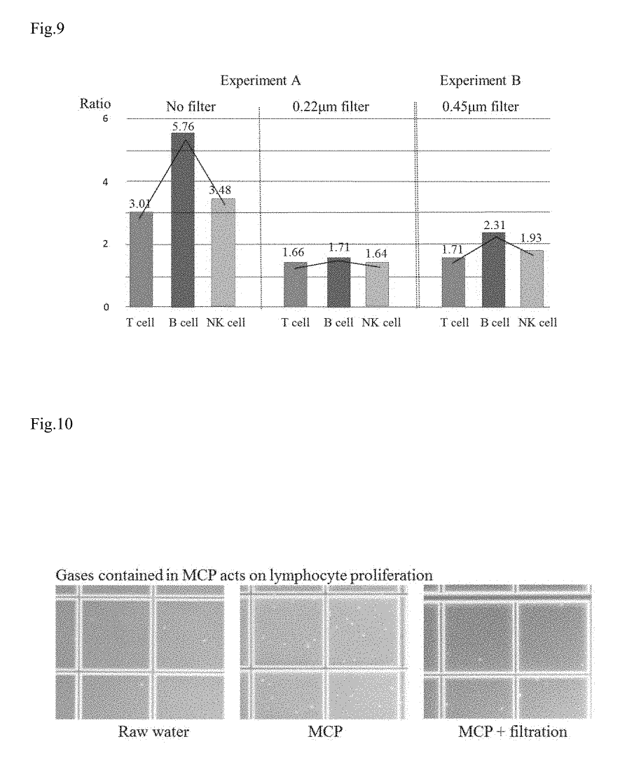

Results of FACS analysis are shown in FIG. 8. In addition, as shown in FIG. 9, when MCP administration group was compared with DW administration group which is assumed 1, all of T cells, B cells and NK cells increased.

There is not so much difference about increase rate of cell number respectively, but among those, increase rate of B cells group which are administered with MCP was large. By this example, MCP of the present invention was shown to be able to be used as injection agent.

### Example 7

In this experiment, 3 kind of water, (1) raw water: raw water for MCP preparation, (2) MCP, (3) MCP+0.22 micrometer filter: 0.22 micrometer filtrate of MCP were administered to a rat by oral administration and intraperitoneal injection (1 ml×2/day, morning, night) and comparison of quantity of each fraction were carried out in blood lymphocytes of a rat after rearing for 7 days.

In observation 7 days after starting experiment, any individuals do not show symptoms such as swelling of stomach or diarrhea by overdose administration of injection and were fine.

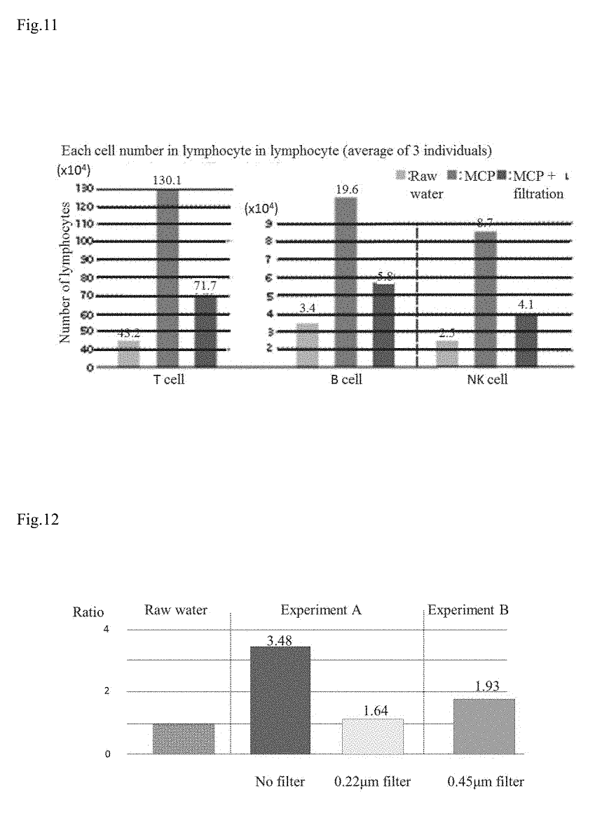

Results (7th day) Significant increase of lymphocyte number was observed when (2) and (3) were administered at the time of measurement under microscope (FIG. 10). From this, it becomes clear that MCP has action to increase lymphocyte number in blood. Specific total lymphocyte number is 50.2×10⁴ in (1), 161×10⁴ in (2) and 8.3×10⁴ in (3). When (1) is assumed 1, ratios are 3.21 in (2) and 1.66 in (3). That is, increase rate of lymphocyte number in case of administration of MCP is 3.21% in average of 3 individuals used in the experiment. Number of T cells, B cells and NK cells in lymphocyte in case of administration of 3 kinds of water, and ratio of each cell number in (2) and (3) administration group when cell number in (1) administration group is assumed 1 are shown in FIG. 11 and FIG. 12. Ratio of number of T cells, B cells and NK cells according to FIG. 12 was similar to the ratio known in ordinary rats. According to FIG. 12, increase rates of these 3 kinds of cell number compared to DW administration group, increase rate of B cell is larger than other 2 kinds of cells. Among each cell fraction of lymphocyte, ratio of (2) and (3) when (1) is assumed 1 are shown in FIG. 12. According to FIG. 12, increase rate of NK cells in (2) is 348% in average of 3 individuals which were counted. Comparison of the results of experiment (1) and (2) In either experiment, in case of MCP administration, all of T cells, B cells and Nk cells increased in blood compared to administration of water without addition of gases (DW or raw water for MCP preparation). This shows gases which are core ingredient of MCP have proliferation promotion effect of lymphocytes. In addition, there showed not so large difference in increase ratio of cell number respectively, but among those, there showed a feature that increase ratio of B cell was large.

### Example 8

### Experiment B

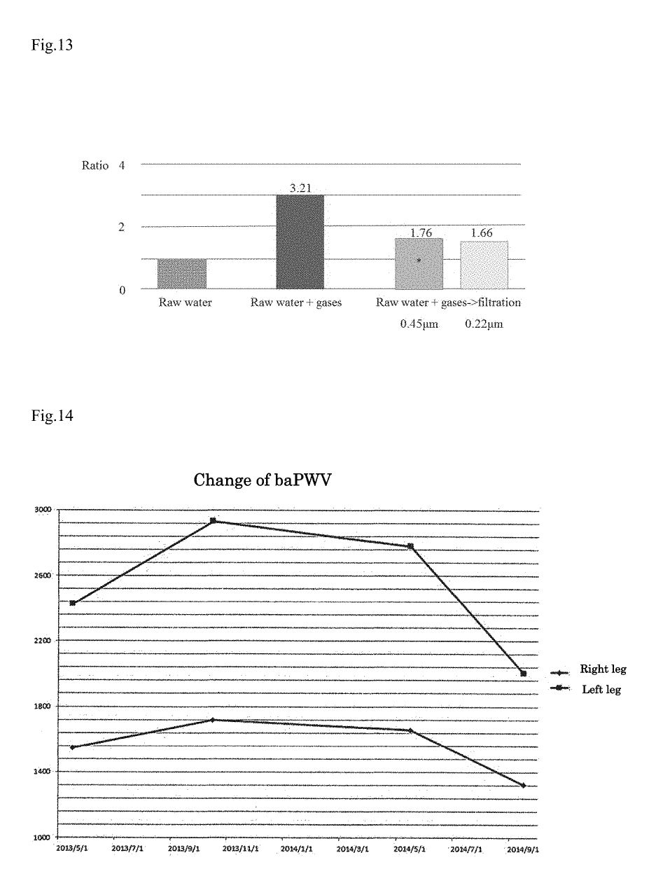

(1) Raw water (raw water for MCP preparation)
(2) Raw water+gas+0.45 micrometer filter
(3) Raw water+gas+0.22 micrometer filter
In experiment B, plot of raw water without filtration, 3.21 fold of total lymphocyte number than that of raw water (FIG. 13). This means growth quantity became more than 3 fold. In contrast, about medium with filtration of raw water+gas, 1.76 fold increase of growth quantity in 0.45 micrometer filter and 1.66 fold increase of growth quantity in 0.22 micrometer filter were observed. These results may be caused by removal of microbubbles by filtration. Diameters of gases are 10 nm to 30 nm.

### Example 9

Effect on gas administration to T cells, B cells and NK cells was measured. In no filter group, B cells growed best and subsequently, NK cells and T cells (FIG. 9).

### Example 10

NK cell number was compared about individuals to which raw water+gas was added. The results are shown in FIG. 12. As shown in FIG. 12, about 3.5 fold growed in no filter. In case with filter, 1.93 fold growth in 0.45 micrometer filter, 1.64 fold growth in 0.22 micrometer filter were shown.

### Example 11

Effect on obstructive arteriosclerosis of both lower limb of 81 years old woman From 2011 spring, she went to hospital for treatment of gonarthrosis with osteonecrosis of both knees and from autumn of 2013, edema mainly to the right leg appeared. In February of 2014, she entered the hospital for right leg thrombus vasculitis, and thrombus dissolving therapy was done. From March of 2014 drinking mitochondria water by 500 ml/day was started.

As a result, reduction of edema and improvement of inflammation were observed. In change of ABI value (ankle-brachial pressure index) which is an arteriosclerotic index, left side value was improved after taking mitochondria water.

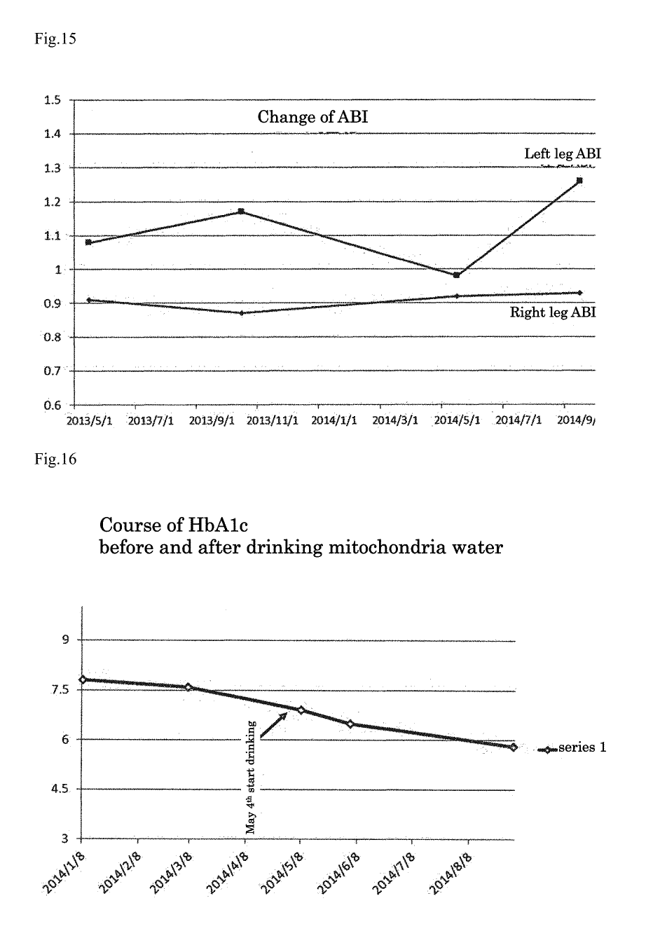

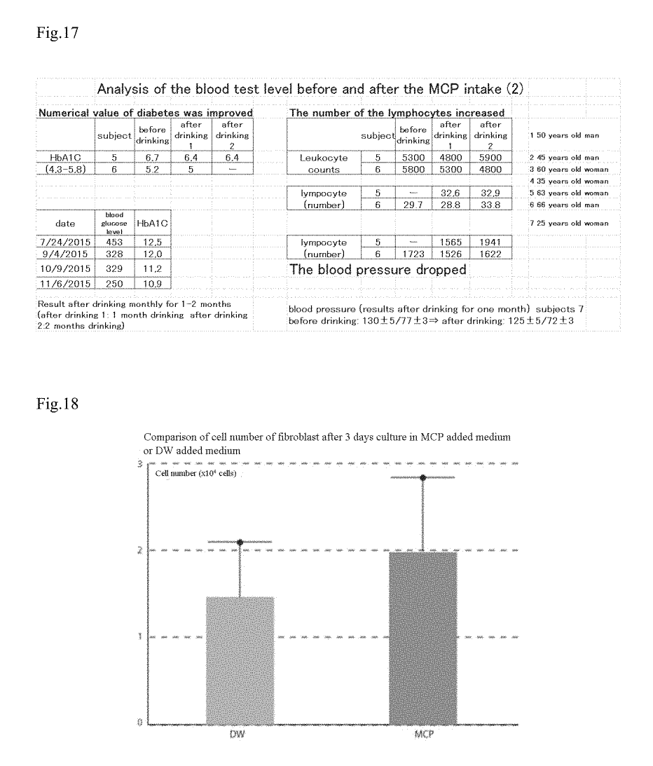

About change of baPWV (brachial-ankle Pulse wave velocity which is an index to evaluate artery compliance), both are improved (refer to table 3 and FIG. 14 to 15).
As there is no anticoagulant as a coadministered drug since before drinking mitochondria water in March, and from May, administration of anti-platelet aggregation agent was started and it is thought that improvement of baPWV is more likely to be influenced by mitochondria water. That is, it is suggested that the effect of mitochondria effect is related to arteriosclerosis improvement of this case

TABLE 3

| | right | left ankle | right | left | right | |
|---|---|---|---|---|---|---|
| | ankle UT | UT | baPWV | baPWV | ABI | left ABI |
| 2013 May 24 | 192 | 185 | 1546 | 2425 | 0.91 | 1.08 |
| 2013 Oct. 4 | 230 | 209 | 1719 | 2929 | 0.87 | 1.17 |
| 2014 May 1 | 181 | 131 | 1657 | 2780 | 0.92 | 0.98 |
| 2014 Sep. 5 | 179 | 149 | 1325 | 2004 | 0.93 | 1.26 |

Ut = Upstroke time ms
PWV = pulse wave velocity
ABI = ankle systolic blood pressure/upper arm systolic blood pressure

### Example 12

Subject: 81 years old woman type II diabetes
February 2008 experience an onset of type II diabetes

Oral medicine and living guidance were focused on and treatment progress was examined.

As oral medicines, sulfonyl preparation, Daonil 1.25 mg (3 tablets 3 after meal) and hyperglycemia improvement agent after a meal, Basen 0.2 mg (3 tablets, 3 before meal) were administered internally.
Hyperlipidemia, a slight renal function drop were recognized as complications.
For hyperlipidemia, 10 mg of atorvastatin calcium formulation was orally administered before sleeping.

Effect of Administration of Mitochondria Water

Thrombolytic therapy was carried out by intravenous Instillation of urokinase in the hospital from May 8^{th} to 16^{th}, and further September 1^{st} to 6^{th}. Until present including above period taking 500 ml/day of mitochondria water was continued. The value of HbA1c (what hemoglobin having role to carry oxygen intracorporeally in a red blood cell and glucose in blood are bound) is dramatically improved from 7.8 on Jan. 8, 2014 before taking to 5.8 on Sep. 1, 2014 (table 4 and FIG. 16). Quantity of oral administration has been the same and there have been no change for 4 years. Type II diabetes was improved by mitochondria water.

TABLE 4
Progress of HbA1c before and after
mitochondrial water drinking

| | |
|---|---|
| 2014 Jan. 8 | 7.8 |
| 2014 Mar. 7 | 7.6 |
| 2014 May 8 | 6.9 |
| 2014 Jun. 4 | 6.5 |
| 2014 Sep. 1 | 5.8 |

### Example 13

MCP drinking test was conducted for human for the aim of elucidating effect which MCP gives to living body when human drinks. Subjects are as follows.
1. 50 years old man, 2. 45 years old man, 3. 60 years old woman, 4. 35 years old woman, 5. 63 years old woman, 6. 66 years old man, 7. 25 years old woman

Each participant had drunk 0.5-1.0 L/day of MCP for 4 weeks living a life without change from usual life. Participants are blood-collected before starting to drink and again blood was collected 4th week and 8th weeks after starting to drink. Test was conducted about items of leucocytes, liver function, lipid-related and diabetes-related respectively. As a result, numerical value of wide range item such as liver function, cholesterol level, triglyceride levels and diabetes-related levels etc. were improved. Improvement of numerical value is significant about abnormal level but about the normal level, an improvement trend to ideal numerical value was observed. Especially, item which significant improvement of numerical value was shown is cholesterol level and all subjects tested showed improvement. In addition, about HDL value, as the value of subject 6 became 45 who had never exceeded 40 at the time of medical examination for the past 20 years, MCP has a possibility to contribute to the increase of HDL levels for which there is no specific medicine (table 5).

TABLE 5

| | | before | | | | before | after | after |
|---|---|---|---|---|---|---|---|---|
| | subject | drinking | after drinking | | subject | drinking | drinking 1 | drinking 2 |
| GPT | 1 | 75 | 72 | total | 1 | 182 | 151 | |
| | | | | cholesterol | | | | |
| (4-37) | 2 | 63 | 42 | (140-199) | 2 | 90 | 80 | |
| | 3 | 63 | 54 | | 5 | 206 | 176 | 176 |
| | 4 | 61 | 38 | | 6 | 180 | 166 | 158 |
| | 5 | 29 | 23 | | | | | |
| | 6 | 23 | 19 | HDL | 5 | 56 | 43 | 52 |
| | | | | (40-74) | 6 | 37 | 45 | 42 |
| γ-GTP | 1 | 182 | 151 | | | | | |
| (9-50) | 2 | 117 | 96 | LDL | 5 | 110 | 88 | 94 |
| | 5 | | | (120 or less) | 6 | 123 | 108 | 104 |
| | 6 | 17 | 15 | | | | | |
| | | | | LDL/HDL | 5 | 2.0 | 2.0 | 1.8 |
| GOT | 1 | 63 | 54 | (Less than 2.0 | 6 | 3.3 | 2.4 | 2.47 |
| | | | | are desireable) | | | | |
| (9-32) | 2 | 56 | 54 | | | | | |
| | 5 | 26 | 21 | neutral fat | 1 | 156 | 172 | |
| | 6 | 23 | 19 | (30-149) | 2 | 192 | 179 | |
| | | | | | 5 | 158 | 218 | 151 |
| | | | | | 6 | 77 | 62 | 59 |

### Example 14

Effect on Plants were Examined Using Japanese Mustard Spinach (Brassica rapa Var. perirdis, Wakami)

Cultivation period Mar. 7, 2014 to Apr. 7, 2014 (32 days)

Methods: 30 L of dechlorinated water was poured into a container and fertilizer for hydroponics, tankmix F&B (table 6, OAT Agrio co., Ltd.) was dissolved. Cultivation solution was adjusted to EC 2.4 and aeration was done for 24 hrs during cultivation period.

Seeds were sowed in a urethane, 10 days after raising seedlings in growth chamber, 9 individuals were planted in each container and 3 repetition (3 containers) examinations were conducted. They were harvested 3 weeks after fix planting.

### Treated Plot Control Plot, Air Microbubble Plot, APAS+MB Plot

In air MB plot, air MB is generated for 20 min. during culture, and in APAS+MB plot, air which is made to MB is generated for 20 min. 3 times a week after fix planting (total 6 treatments).

Examined items height of a plant, maximum leaf length, maximum root length, above ground part fresh weight, subterranean part fresh weight, above ground part dry weight, subterranean part dry weight.

Results are shown in table 7. For height of a plant, maximum leaf length, maximum root length, above ground part fresh weight and subterranean part fresh weight, no significant difference was observed. However, above ground part fresh weight and subterranean part fresh weight of APAS+MB plot increased significantly compared with control plot and MB plot. Growth promotion effect was observed in APAS+MB.

TABLE 6
Tankmix F&B heavy undiluted solution ingredient quantity (g/100 L)

| | | calculated from guaranteed ingredient | | | | | | calculated from combination ingredient | | | | |
|---|---|---|---|---|---|---|---|---|---|---|---|---|
| | | nitrogen | phosphoric | | | | | | | | | Calcium |
| Tank mix | volume (l) | (AN/NN) | acid | potassium | magnesia | Manganese | boron | iron | copper | zinc | Molybdenum | (CaO) |
| F & B | 100 | 2700 | 1870 | 3920 | 700 | 33 | 17 | 44 | 0.4 | 1.3 | 1.1 | 2200 |
| | | (121/2436) | | | | | | | | | | |

(From the description of OAT Agrio co., Ltd. HP)

TABLE 7

| | | maximum leaf | maximum root |
|---|---|---|---|
| test section | plant hight (cm) | length (cm) | length (cm) |
| control | 16.85 ± 0.34 | 23.76 ± 0.33 | 46.72 ± 2.70 |
| section | | | |
| MB section | 16.98 ± 0.47 | 23.19 ± 0.32 | 45.17 ± 2.18 |
| APAS + MB | 17.41 ± 0.28 | 23.32 ± 0.26 | 44.07 ± 2.05 |
| section | | | |

| | | | | Subterranean |
|---|---|---|---|---|
| | Above ground | Above ground | Subterranean | part dry |
| test | part fresh | part dry | part fresh | weight |
| section | weight (g) | weight (g) | weight (g) | (g) |
| control | 22.91 ± 1.04 b | 1.50 ± 0.06 | 1.82 ± 0.09 b | 0.08 ± 0.02 |
| section | | | | |
| MB | 23.53 ± 1.06 b | 1.58 ± 0.06 | 1.81 ± 0.08 b | 0.08 ± 0.01 |
| section | | | | |
| APAS + | 27.00 ± 1.34 a | 1.71 ± 0.10 | 2.16 ± 0.16 a | 0.11 ± 0.01 |
| MB | | | | |
| section | | | | |

Different alphanumeric character means significant at a 5% standard (Fisher's Least Significant Difference Method)
Numerical value following ± shows standard error (Fisher's Least Significant Difference Method)

### Example 15

### Experiment on Proliferation Promotion Action of Fibroblast Cells in MCP Added Medium

### Preparation of Medium

1. Control

| | | |
|---|---|---|
| DW | 285 | g |
| DMEM powder | 3 | g |
| NaHCO3 | 1.11 | g |
| Total | 300 | ml |

After mixing the above and it was filter-sterilized by 0.45 micrometer filter. 10% FBS (fetal bovine serum) was prepared by 10 ml or by 50 ml and 1/10 volume were added to medium.

2. 1 × MCP

| | | |
|---|---|---|
| MCP | 47.5 | ml |
| DMEM powder | 0.5 | g |
| NzHCO3 | 0.185 | g |
| Total | 50 | ml |

After mixing the above, it was filtered. 9 ml of above solution (DMEM (MCP) and 1 ml of FBS were mixed and used for culture.

3. 2 × DMEM + MCP

| | | |
|---|---|---|
| DW | 95 | ml |
| DMEM powder | 2 | g |
| NaHCO3 | 0.74 | g |
| Total | 100 | ml |

After mixing the above, filtered. After that, they were diluted twice with MCP

| | |
|---|---|
| Filtrated MCP | 4.5 ml |
| 2 × DMEM | 4.5 ml |
| FBS | 1 ml |
| Total | 10 ml |

Cryopreserved mouse fibroblast (MEF) cells were initiated, were subcultured more than 3 times and were used for the experiment. As above, they were cultured in DMEM (High Glucose) prepared with final concentration of 10% FBS. Medium was exchanged once in 3 days and subculture was done at the time of confluent.

The experiment, at first, in 12 well plate, MEF cells were subcultured to become 10,000 cells in 1 well. Next day of subculture, MEF cells were washed once with PBS and were added with DMEM (High Glucose) prepared with final concentration of 10% FBS and Penicillin/Streptomycin using DW or MCP. Analysis was conducted 3^{rd} day assuming the day MCP was added 0^{th} day. Medium was changed every day after MCP was added.

For counting cell numbers, 0.25% of trypsin was added and only viable cells were counted by trypan blue staining.

Culture experiment was conducted in duplicate and 2 days and 8 days culture were conducted. From the result of these two times of experiments, it was suggested that MCP has proliferation promotion effect of fibroblast cells (FIG. 18). Their growth rates were 125 to 200% compared with the case adding DW to medium (table 8).

TABLE 8

| | | 1. DMEM | | | 2. MCP-DMEM | | | 3. 2 × DMEM + MCP | | |
|---|---|---|---|---|---|---|---|---|---|---|
| | | 1-1 | 1-2 | Ave. | 2-1 | 2-2 | Ave. | 3-1 | 3-2 | Ave. |
| 2 days | 1st/ml | 1.75 | 1.63 | | 2 | 2.28 | | 2.44 | 2.78 | |
| culture | 2nd/ml | 1.72 | 1.75 | | 2.19 | 2.06 | | 2.53 | 2.97 | |
| | Ave./ml | 1.73 | 1.69 | 1.71 | 2.09 | 2.17 | 2.13 | 2.48 | 2.88 | 2.68 |
| 8 days | 1st/ml | 0.84 | 0.65 | | 1.15 | 1.13 | | 1.11 | 1.23 | |
| culture | 2nd/ml | 0.81 | 0.73 | | 1 | 0.9 | | 0.99 | 1.11 | |
| | Ave./ml | 0.83 | 0.69 | 0.76 | 1.08 | 1.01 | 1.04 | 1.05 | 1.17 | 1.11 |

A few dispersion observed in experimental results, are supposed to be caused by difference of cellular lot used in the experiment or to be caused by fluctuation of experimental conditions

Fibroblast cells have function to produce collagen, elastin, and hyaluronic acids in the epidermis. In addition, they have function of forming dermis making collagen to fiber bundle. Exposed to a large quantity of ultra-violet light or accelerating the aging process, fibers in regularity can not be generated and a wrinkle or slack occurs. MCP with proliferation promotion effect of fibroblast is expected to show effective suppression effect to aging of skin.

### Example 16

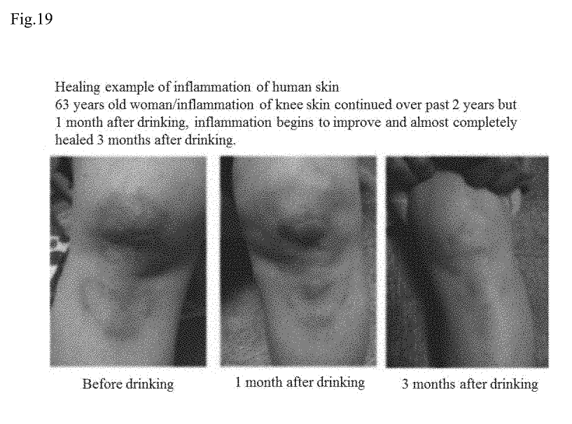

The 63 years old woman who had inflammation on a knee had continued inflammation for about 2 years but by drinking 500 ml to 1 L every day, inflammation was significantly improved after 1 month and inflammation almost disappeared after 3 months (FIG. 19). Quantity of drinking MCP is preferably more than 150 ml a day, more preferably, more than 300 ml, more preferably, more than 500 ml, more preferably, more than 1 L. All the water to take per day may be MCP.

### Example 17

After letting a pet drink MCP as water to drink every day for 30 days, as shown in table 9, Index numerical value of the disease is improved.

TABLE 9

| | | | toy poodle | | shih-tzu | | Shiba Inu | | yorkshire terrier | |
|---|---|---|---|---|---|---|---|---|---|---|
| | | | sex | age | sex | age | sex | age | sex | age |
| | | | male | 6 | female | 12 | female | 7 | female | 16 |
| | The disease that | normal | before | after | before | after | before | after | before | after |
| items | is assumed | value | drinking | drinking | drinking | drinking | drinking | drinking | drinking | drinking |
| GLU | diabetes | 55-120 | 109 | 104 | 97 | 91 | 91 | 108 | 91 | 97 |
| BUN | kidney | 8-30 | 15 | 14 | 19 | 14 | 16 | 20 | 37 | 22 |
| | dysfunction | | | | | | | | | |
| CRE | kidney | 0.5-2.0 | 1.0 | 0.8 | 1.1 | 1.2 | 1.3 | 1.2 | 0.9 | 1.1 |
| | dysfunction | | | | | | | | | |
| LDH | liver, kidney, | 0-263 | 102 | 100- | 100- | 100- | 199 | 100- | 100- | 131 |
| | muscle cell | | | | | | | | | |
| | necrosis, tumor | | | | | | | | | |
| CA | Thyroidal | 8.7-11.4 | 11.4 | 10.6 | 12.8 | 12.1 | 9.8 | 10.0 | 11.7 | 9.7 |
| | abnormality, | | | | | | | | | |
| | renal | | | | | | | | | |
| | insufficiency | | | | | | | | | |
| TG | hyperlipidemia | 7.7-53.2 | 93 | 81 | 48 | 41 | 27 | 94 | 45 | 37 |
| CPK | heart muscle, | 40-151 | 236 | 151 | 90 | 83 | 85 | 86 | 80 | 69 |
| | skeletal muscle | | | | | | | | | |
| | necrosis | | | | | | | | | |
| AMY | Paricreatitis, | 360-1800 | 336 | 329 | 1110 | 1357 | 1645 | 323 | 2500+ | 2500+ |
| | renal | | | | | | | | | |
| | insufficiency | | | | | | | | | |
| | hepatitis | | | | | | | | | |

What is claimed is:

1. A method for activating mitochondria comprising:
the step of producing a composition containing 0.45-0.55 ppm of hydrogen, 10-12.5 ppm of oxygen and 7-8 ppm of nitrogen in a form of microbubbles with 30 micrometers or less in a diameter and/or in a form of nanobubbles with 30 micrometers or less in a diameter, and
the step of treating a cell with the composition.

2. A method according to claim 1, further comprising the step of promoting cell proliferation.

3. A method according to claim 2, wherein the cell is an animal cell.

4. A method according to claim 3, wherein the animal cell is an immune-system cell.

5. A method according to claim 1, further comprising the step of preserving.

6. A method according to claim 5, wherein the step of preserving is the step of cryopreserving.

7. A method according to claim 1, wherein the cell is that of a food.

8. A method according to claim 1, further comprising the step of treating diabetes.
